(19) United States
(12) Patent Application Publication
(10) Pub. No.: US 2026/0144511 A1
(43) Pub. Date: May 28, 2026

(54) SYSTEM OF DETERMINING PATIENT-SPECIFIC ANGULAR RANGE FOR DIGITAL BREAST TOMOSYNTHESIS AND METHOD THEREOF

(71) Applicant: KOREA ADVANCED INSTITUTE OF SCIENCE AND TECHNOLOGY, Daejeon (KR)

(72) Inventors: Seungryong CHO, Daejeon (KR); Seoyoung LEE, Daejeon (KR); Subong HYUN, Daejeon (KR); Donghyun KIM, Daejeon (KR)

(73) Assignee: KOREA ADVANCED INSTITUTE OF SCIENCE AND TECHNOLOGY, Daejeon (KR)

(21) Appl. No.: 19/198,900

(22) Filed: May 5, 2025

(30) Foreign Application Priority Data
Nov. 22, 2024 (KR) 10-2024-0168384

(51) Int. Cl. A61B 6/50; A61B 6/00; A61B 6/02

(52) U.S. Cl. CPC A61B 6/502; A61B 6/025; A61B 6/5217; A61B 6/542; A61B 6/545

(57) ABSTRACT

A digital breast tomosynthesis (DBT) method includes: disposing a breast of an examination subject between an X-ray irradiator and an X-ray detector; acquiring first image data by emitting X-rays toward the breast of the examination subject; analyzing the first image data to estimate a type of breast lesion by using a pre-trained AI image diagnosis portion; determining an angular range for performing DBT according to the estimated type of breast lesion; performing the DBT in which the X-ray irradiator rotates around the breast within the determined angular range and emits X-rays toward the breast at every predetermined angular interval, to acquire second image data; and reconstructing the second image data to generate a tomosynthesis image, wherein a first dose, which is a total X-ray dose in acquiring the first image data, is smaller than a second dose, which is a total X-ray dose in performing the DBT.

### CROSS-REFERENCE TO RELATED APPLICATION

This application claims priority under 35 U.S.C. § 119 to Korean Patent Application No. 10-2024-0168384, filed on Nov. 22, 2024, in the Korean Intellectual Property Office KIPO, the disclosure of which is incorporated by reference herein in its entirety.

### BACKGROUND OF THE INVENTION

### (1) Field of the Invention

The present disclosure relates to a digital breast tomosynthesis system capable of determining a patient-specific angular range for digital breast tomosynthesis and a method thereof.

### (2) Description of Related Art

Digital breast tomosynthesis (hereinafter, “DBT”) is a technology proposed to overcome the shortcomings of conventional digital mammography. With the DBT, the breast may be scanned from a plurality of angles to reconstruct the inside of the breast into a virtual three-dimensional image.

In conventional mammography, customizable variables such as the voltage and current of the X-ray exposure were limited. However, DBT may provide additional variables such as the range of imaging (e.g., scanning) angles. Studies have shown that detection performance for microcalcifications is better in imaging with a narrow-angular range, while detection of masses is better in imaging with a wide-angular range. However, considering the radiation exposure to the patient, a single angular range should be chosen for the imaging process. In conventional DBT systems, a single angular range was used without considering the different characteristics of each patient.

Accordingly, there is a need for a system and method that selects an appropriate scanning angular range based on the specific characteristics of the patient.

### SUMMARY

The present disclosure is directed to a DBT system and method capable of determining a patient-specific angular range for DBT.

The present disclosure is directed to a DBT system and method capable of performing DBT in an angular range more suitable for each patient.

The present disclosure is directed to a DBT system and method capable of setting a patient-specific angular range for DBT by utilizing data acquired during a scout scan process for X-ray automatic exposure control.

According to an embodiment of the present disclosure, a digital breast tomosynthesis (DBT) method includes: disposing a breast of an examination subject between an X-ray irradiator and an X-ray detector; acquiring first image data by emitting X-rays by the X-ray irradiator toward the breast of the examination subject; analyzing the first image data to estimate a type of breast lesion of the examination subject by using at least one of an artificial intelligence (AI) image diagnosis portion and an artificial neural network pre-trained based on breast image data categorized by breast lesion type; determining an angular range for performing DBT according to the estimated type of breast lesion; performing the DBT in which the X-ray irradiator rotates around the breast of the examination subject within the determined angular range and emits X-rays toward the breast at every predetermined angular interval, to acquire second image data corresponding to a plurality of angles; and reconstructing the second image data acquired at the plurality of angles to generate a tomosynthesis image, wherein a first dose, which is a total X-ray dose used in acquiring the first image data, is smaller than a second dose, which is a total X-ray dose used in performing the DBT.

In some embodiments, wherein in a case where the estimated type of breast lesion of the examination subject is a mass, the determined angular range may be a first angular range, in a case where the estimated type of breast lesion of the examination subject is microcalcification, the determined angular range may be a second angular range, in a case where the estimated type of breast lesion of the examination subject is a lesion other than mass or microcalcification, or where the estimated type of breast lesion of the examination subject is undetected, the determined angular range may be a predetermined angular range selected between the first angular range and the second angular range, and the first angular range may be wider than the second angular range.

In some embodiments, the first angular range may be in a range of 40 to 60 degrees, and the second angular range may be in a range of 5 to 25 degrees.

In some embodiments, in a case where the estimated type of breast lesion of the examination subject is a mass, the determined angular range may be a first angular range, in a case where a mass is undetected in the estimated type of breast lesion of the examination subject, the determined angular range may be a second angular range, and the first angular range may be wider than the second angular range.

In some embodiments, the DBT method may further include performing automatic exposure control (AEC) for the X-rays emitted during the performing the DBT, based on the first image data.

In some embodiments, the DBT method may further include determining at least one of the second dose, voltage, current, and exposure time of the X-rays emitted during the performing the DBT, based on the first image data.

In some embodiments, the first dose may be 50% or less of the second dose.

In some embodiments, the at least one of the pre-trained AI image diagnosis portion and the pre-trained artificial neural network may include at least one of an AI image diagnosis module, a computer-aided diagnosis (CAD) module, a machine learning network, a deep learning network, a machine learning/deep learning classification model, and a machine learning/deep learning regression model.

In some embodiments, the at least one of the pre-trained AI image diagnosis portion and the pre-trained artificial neural network may be trained to receive the first image data and estimate the type of breast lesion of the examination subject based on the first image data.

According to an embodiment of the present disclosure, a digital breast tomosynthesis (DBT) system includes: an X-ray irradiator; an X-ray detector disposed to face the X-ray irradiator; a memory configured to store computer-readable instructions for controlling the X-ray irradiator and the X-ray detector to generate a tomosynthesis image; and a processor electrically connected to the memory and implemented to execute the instructions, wherein the processor, upon executing the instructions, is configured to: acquire first image data by the X-ray irradiator emitting X-rays toward the breast of the examination subject disposed between the X-ray irradiator and the X-ray detector; analyze the first image data to estimate a type of breast lesion of the examination subject by using at least one of an artificial intelligence (AI) image diagnosis portion and an artificial neural network pre-trained based on breast image data categorized by breast lesion type; determine an angular range for performing DBT according to the estimated type of breast lesion; perform the DBT in which the X-ray irradiator rotates around the breast of the examination subject within the determined angular range and emits X-rays toward the breast at every predetermined angular interval, to acquire second image data corresponding to a plurality of angles; and reconstruct the second image data acquired at the plurality of angles to generate a tomosynthesis image, and a first dose, which is a total X-ray dose used in acquiring the first image data, is smaller than a second dose, which is a total X-ray dose used in performing the DBT.

In some embodiments, in a case where the estimated type of breast lesion of the examination subject is a mass, the determined angular range may be a first angular range, in a case where the estimated type of breast lesion of the examination subject is microcalcification, the determined angular range may be a second angular range, in a case where the estimated type of breast lesion of the examination subject is a lesion other than mass or microcalcification, or where the estimated type of breast lesion of the examination subject is undetected, the determined angular range may be a predetermined angular range selected between the first angular range and the second angular range, and the first angular range may be wider than the second angular range.

In some embodiments, in a case where the estimated type of breast lesion of the examination subject is a mass, the determined angular range may be a first angular range, in a case where a mass is undetected in the estimated type of breast lesion of the examination subject, the determined angular range may be a second angular range, and the first angular range may be wider than the second angular range.

In some embodiments, the processor may be further configured to perform automatic exposure control (AEC) for the X-rays emitted during the performing the DBT, based on the first image data.

In some embodiments, the processor may be further configured to determine at least one of the second dose, voltage, current, and exposure time of the X-rays emitted during the performing the DBT, based on the first image data.

A DBT system and method according to an embodiment of the present disclosure may determine a patient-specific angular range for DBT.

A DBT system and method according to an embodiment of the present disclosure may perform DBT in an angular range more suitable for each patient.

A DBT system and method according to an embodiment of the present disclosure may set a patient-specific angular range for DBT by utilizing data acquired during a scout scan process for X-ray automatic exposure control.

### BRIEF DESCRIPTION OF THE DRAWINGS

A more complete appreciation of the present disclosure will become more apparent by describing in detail exemplary embodiments thereof with reference to the accompanying drawings, wherein:

FIG. 1 is a view schematically illustrating configuration of a DBT system according to an embodiment of the present disclosure,

FIG. 2A is a view schematically illustrating a process in which an imaging portion of a DBT system according to an embodiment of the present disclosure performs a scout imaging of an examination subject,

FIG. 2B is a view schematically illustrating a process in which an imaging portion of a DBT system according to an embodiment of the present disclosure performs DBT on an examination subject within a first angular range,

FIG. 2C is a view schematically illustrating a process in which an imaging portion of a DBT system according to an embodiment of the present disclosure performs DBT on an examination subject within a second angular range,

FIG. 3A is a view for explaining a process in which a first lesion type is imaged within the first angular range according to an embodiment of the present disclosure,

FIG. 3B is a view for explaining a process in which a second lesion type is imaged within the second angular range according to an embodiment of the present disclosure,

FIG. 4 is a flowchart illustrating a DBT method according to an embodiment of the present disclosure, and

FIG. 5 is a flowchart illustrating a DBT method according to another embodiment of the present disclosure.

### DETAILED DESCRIPTION

Hereinafter, embodiments of the present disclosure will be described in detail with reference to the accompanying drawings, such that those skilled in the art may easily practice the invention. The present invention may be implemented in various different forms and is not limited to the embodiments described herein.

The drawings are schematic and not drawn to scale. The relative dimensions and proportions of the parts in the drawings are exaggerated or reduced in size for clarity and convenience in the drawings, and any dimensions are merely exemplary and not limiting. Furthermore, the same reference numerals are used in two or more drawings to denote similar features of identical structures, elements, or parts.

The embodiments of the present invention specifically illustrate the ideal embodiments of the invention. As a result, various modifications of the drawings are expected. Therefore, the embodiments are not limited to the specific forms of the illustrated areas and include modifications of forms due to manufacturing, for example.

In addition, all technical and scientific terms used in this specification have meanings generally understood by those skilled in the art to which the present invention belongs, unless otherwise defined. All terms used in this specification have been selected for the purpose of more clearly explaining the present invention and have not been selected to limit the scope of rights according to the present invention.

Additionally, expressions such as “including,” “having,” or “comprising” used in this specification should be understood as open-ended terms, implying the possibility of including other embodiments, unless otherwise stated in the phrase or sentence in which the expression is included. Furthermore, expressions in the singular form described in this specification may include plural meanings unless otherwise stated, and this also applies to expressions in the singular form described in the claims.

Additionally, the terms “first,” “second,” “third,” and the like may be used in this specification to describe various components, but these components are not limited by the terms. The terms are used for the purpose of distinguishing one component from another. For example, without departing from the scope of rights of the present invention, a first component may be referred to as a second or third component, and the like, and similarly, a second or third component may be referred to interchangeably.

Herein, digital breast tomosynthesis (DBT) involves reconstructing a plurality of projection images into thin slices to facilitate visual evaluation. The projection images of a breast may be acquired by a digital detector with a radiation source (e.g., X-ray source) that moves within a predetermined angle, and the acquired images may be reconstructed in a manner similar to computed tomosynthesis (CT) or magnetic resonance imaging (MRI).

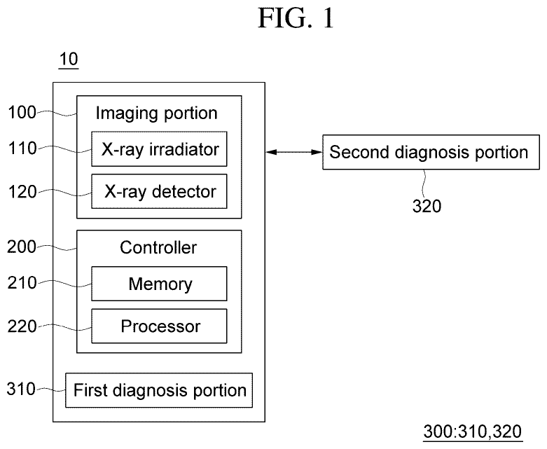

Hereinafter, a DBT system 10 according to an embodiment of the present disclosure will be described with reference to FIG. 1. FIG. 1 is a view schematically illustrating configuration of the DBT system 10 according to an embodiment of the present disclosure.

The DBT system 10 according to an embodiment of the present disclosure may include an imaging portion 100 and a controller 200 configured to control the imaging portion 100. The DBT system 10 according to an embodiment of the present disclosure may further include a diagnosis portion 300. The controller 200 may be electrically connected to or may transmit and receive signals through a network, a server, communication, and the like with the imaging portion 100 and the diagnosis portion 300 and control the operation of the imaging portion 100 and the diagnosis portion 300.

The imaging portion 100 may include an X-ray irradiator (e.g., X-ray source) 110 and an X-ray detector 120 disposed to face the X-ray irradiator.

The X-ray irradiator 110 may emit X-rays toward the X-ray detector 120. For example, the X-ray irradiator 110 may include an X-ray tube (not illustrated) that receives tube voltage and tube current from a generator (not illustrated) to generate X-rays. In addition, the X-ray irradiator 110 may include an emission port that may allow X-rays generated by the X-ray tube to be emitted in a predetermined direction and within a predetermined angular range toward, for example, the X-ray detector 120. The X-ray irradiator 110 according to an embodiment of the present disclosure is not particularly limited and may include any known X-ray irradiator.

The X-ray detector 120 may detect X-rays emitted from the X-ray irradiator 110 and having passed through an examination subject so as to output image data. For example, the X-ray detector 120 may detect X-rays having passed through the examination subject and incident on the X-ray detector 120, and generate a signal for image generation and output the signal to the controller 200 or the diagnosis portion 300. The X-ray detector 120 according to an embodiment of the present disclosure is not particularly limited and may include any known X-ray detector. Herein, the examination subject may refer to a patient undergoing a breast examination.

In an embodiment, at least one of the X-ray irradiator 110 and the X-ray detector 120 may be disposed to be movable e.g., rotatable. For example, the X-ray irradiator 110 may emit X-rays toward the examination subject while moving (e.g., rotating) around the examination subject positioned between the X-ray irradiator 110 and the X-ray detector 120. The X-ray detector 120 may be fixedly disposed, or may rotate around the examination subject together with the X-ray irradiator 110 according to the movement of the X-ray irradiator 110. To this end, the imaging portion 100 may include structures such as arms and rails for the movement of the X-ray irradiator 110 and the X-ray detector 120. That is, the X-ray irradiator 110 and the X-ray detector 120 may emit X-rays toward the examination subject while moving around the examination subject and detect the X-rays to acquire an image (e.g., a scan image). However, embodiments of the present disclosure are not limited thereto, and the X-ray irradiator 110 and the X-ray detector 120 may be fixedly disposed, and a chamber (not illustrated) in which the examination subject is located may move (rotate) with respect to the X-ray irradiator 110 and the X-ray detector 120 such that the X-ray irradiator 110 and the X-ray detector 120 may acquire images, and the movement of the chamber (not illustrated) in which the examination subject is located may be controlled by the controller 200.

The controller 200 may be electrically connected to or may transmit and receive signals through a network, a server, communication, and the like with the imaging portion 100 to control the operation and movement of the X-ray irradiator 110 and the X-ray detector 120 of the imaging portion 100, and process the image data received from the X-ray detector 120. In addition, the controller 200 may be electrically connected to or may transmit and receive signals through a network, a server, communication, and the like with the diagnosis portion 300 to control the operation of the diagnosis portion 300, and may control the imaging portion 100 based on breast lesion type estimated by the diagnosis portion 300.

The controller 200 may include a memory 210 and a processor 220 that may be electrically connected to or may transmit and receive signals through a network, a server, communication, and the like with the memory 210 to execute instructions stored in the memory 210. In an embodiment, the DBT system 10 may further include a computer device, a CPU (Central Processing Unit), an operating system, a RAM (Random Access Memory), a ROM (Read Only Memory), an input/output device, an external interface, a server, communication equipment, a bus, and the like for loading and operating the memory 210 and the processor 220 of the controller 200.

The memory 210 may include a computer-readable storage medium and/or a communication medium capable of storing instructions executable by the processor 220. For example, the memory 210 may store computer-readable instructions. The computer storage medium may include any type of storage unit, such as volatile memory, non-volatile memory, and/or other permanent and/or auxiliary computer storage media implemented in any method or technology for storing information, such as computer-readable instructions, data structures, program modules, or other data, removable and non-removable computer storage media. The communication medium may embody data in the form of computer-readable instructions, data structures, program modules, or a modulated data signal such as a carrier wave or other transmission mechanism.

In an embodiment, the memory 210 may store instructions for the X-ray irradiator 110 of the DBT system 10 to emit X-rays and the X-ray detector 120 to detect X-rays. For example, the memory 210 may store at least one of a path along which the X-ray irradiator 110 and the X-ray detector 120 move (rotate), an angular range, and an angular interval (e.g., angular increment, unit, step, etc.) for performing the scanning (imaging) at every angular interval, a dose of X-ray irradiation, voltage, current, exposure time, exposure interval, frequency, and wavelength to emit X-rays, thereby allowing the controller 200 to control the imaging portion 100.

The processor 220 may execute the instructions stored in the memory 210. For example, the processor 220 may execute the instructions stored in the nonvolatile memory of the memory 210 or other permanent and/or auxiliary computer storage medium, thereby reading out the program or data according to the instructions onto the volatile memory of the memory 210 and executing the processing, thereby realizing the control or function of the overall DBT system 10. For example, the processor 220 may control the operation of the imaging portion 100 and the diagnosis portion 300, transmit and receive signals, and process received data by executing instructions stored in the memory 210.

The diagnosis portion 300 may receive and analyze the image data detected by the X-ray detector 120, and may estimate type of breast lesion of the examination subject as indicated in the image data. In an embodiment, the diagnosis portion 300 may be pre-trained based on breast image data categorized by type of breast lesion. Herein, the type of breast lesion may include mass, microcalcification, and other lesions other than mass and microcalcification. Herein, the mass may include lump, cyst, fibroadenoma, breast cancer, hamartoma, fibrocystic change, papilloma, abscess, hyperplasia, and the like formed in the breast.

For example, the diagnosis portion 300 may perform pre-training based on breast image data categorized by type of breast lesion from a confirmed patient group, and analyze the image data detected by the X-ray detector 120 based on the pre-training to estimate type of breast lesion of the examination subject indicated in the image data. For example, the diagnosis portion 300 may estimate a probability value for each type of breast lesion of the examination subject.

In addition, the diagnosis portion 300 may diagnose that it is “normal” or “undetected (e.g., no abnormality detected)” if the breast lesion is not present or undetected in the examination subject. The diagnosis portion 300 may include an artificial intelligence (AI) image diagnosis module, a computer-aided diagnosis (CAD) module, an artificial neural network, a machine learning network, a deep learning network, a machine learning/deep learning classification model, a machine learning/deep learning regression model, and the like that may be trained in advance based on breast image data categorized by type of breast lesion. However, embodiments of the present disclosure are not limited thereto, and the diagnosis portion 300 may use any known AI diagnosis model, algorithm, and method for diagnosing breast lesions based on image data without any particular limitation.

The diagnosis portion 300 may include at least one of a first diagnosis portion 310 included in the DBT system 10 and a second diagnosis portion 320 that is electrically connected to the DBT system 10 from outside the DBT system 10 or may transmit and receive signals via a network, a server, communication, and the like with the DBT system 10. The first diagnosis portion 310 and the second diagnosis portion 320 may receive and analyze the image data detected by the X-ray detector 120, and may output the estimation result of the type of breast lesion of the examination subject shown in the image data to, for example, the controller 200. Herein, the estimated type of breast lesion may include mass, microcalcification, and other lesions other than mass and microcalcification. In addition, the diagnosis portion 300 may diagnose that it is “normal” or “undetected (no abnormality detected)” if a breast lesion is not detected for the examination subject. Although it is illustrated in FIG. 1 that the first diagnosis portion 310 included in the DBT system 10 is a separate configuration from the controller 200, embodiments of the present disclosure are not limited thereto. For example, the first diagnosis portion 310 may be included in the controller 200, and the memory 210 may include instructions that enable the first diagnosis portion 310 to analyze image data and execute a process of estimating the type of breast lesion through the processor 220.

In addition, although not illustrated in the drawings, the DBT system 10 may further include an input portion configured to receive an input from a user such that the user may set or adjust in real time at least one of a path along which at least one of the X-ray irradiator 110 and the X-ray detector 120 moves (rotates), an angular range, and an angular interval based on which the scanning is performed at every angular interval, a dose, voltage, current, exposure time, exposure interval, frequency, and wavelength for X-ray irradiation, thereby allowing the controller 200 to control the X-ray irradiator 110 and the X-ray detector 120. In addition, although not illustrated in the drawings, the DBT system 10 may further include a display portion configured to display and output detected image data, diagnostic information such as estimated type of breast lesion, scout image, tomosynthesis image generated based on the image data, and information thereon.

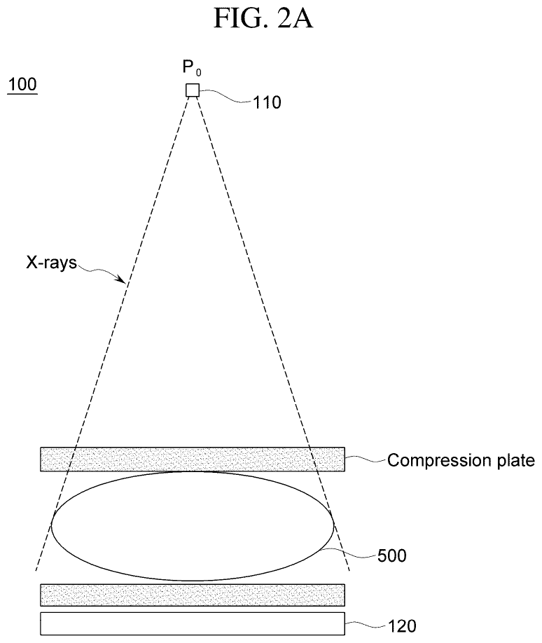

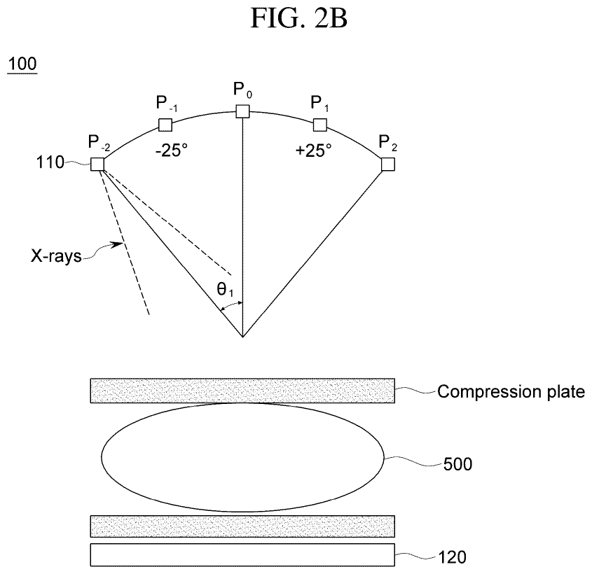

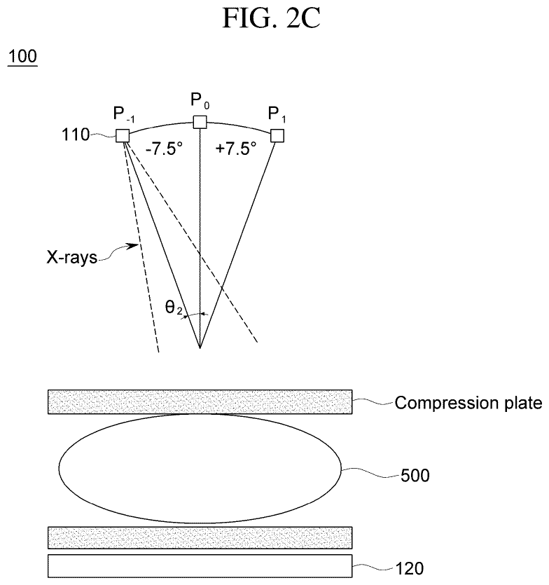

Hereinafter, with reference to FIGS. 1 and 2A to 2C, a process of a DBT system acquiring a tomosynthesis image according to an embodiment of the present disclosure will be described. FIG. 2A is a view schematically illustrating a process in which an imaging portion of a DBT system according to an embodiment of the present disclosure performs scout imaging of an examination subject (e.g., a patient). FIG. 2B is a view schematically illustrating a process in which an imaging portion of a DBT system according to an embodiment of the present disclosure performs DBT on a patient within a first angular range. FIG. 2C is a view schematically illustrating a process in which an imaging portion of a DBT system according to an embodiment of the present disclosure performs DBT on a patient within a second angular range.

Referring to FIGS. 2A to 2C, a breast 500 of an examination subject is located between the X-ray irradiator 110 and the X-ray detector 120. For example, the breast 500 of the examination subject may be compressed vertically (e.g., in an up-down direction) by a pair of compression plates for image capturing (e.g., scanning). In FIGS. 2A to 2C, the breast 500 is illustrated as each breast of the examination subject being imaged separately, but embodiments of the present disclosure are not limited thereto, and both breasts may be imaged at once. For example, for scanning, the examination subject may be located such that a center portion of each breast 500 of the examination subject is located at a center portion of the X-ray detector 120 in a left and right direction of the examination subject. Referring to FIGS. 2A to 2C, the X-ray irradiator 110 may emit X-rays to an area within a range indicated by a dotted line at each position P₋₂, P₋₁, P₀, P₁, P₂ set in advance for X-ray irradiation, but embodiments of the present disclosure are not limited thereto, and the X-ray irradiator 110 may emit X-rays to an area wider or narrower than the dotted line area of FIGS. 2A to 2C.

In an example, the X-ray irradiator 110 may emit X-rays from above the breast 500 of the examination subject toward the breast 500, and the X-ray detector 120 may be disposed below the breast 500 to detect the X-rays that have passed through the breast 500. Although not illustrated, in another example, the X-ray irradiator 110 may emit X-rays from below the breast 500 of the examination subject toward the breast 500, and the X-ray detector 120 may detect the X-rays that have passed through the breast 500 from above the breast 500. For example, the X-ray irradiator 110 may emit X-rays toward the breast 500 of the examination subject at every predetermined angular interval while moving around the breast of the examination subject along an arc that passes above or below the breast 500 from one side toward an opposite side of the breast 500. For example, the X-ray irradiator 110 may emit X-rays toward the breast 500 of the examination subject at every predetermined angular interval while rotating around the X-ray detector 120 (e.g., the breast 500 of the examination subject) along an arc with respect to an axis that passes from the back to the front of the examination subject.

Referring to FIGS. 1 and 2A, in an embodiment, the imaging portion 100 may emit X-rays toward the breast 500 of the examination subject to acquire first image data. The first image data may also be referred to as scout image data, preliminary image data, or reconnaissance image data. In an embodiment, the X-ray irradiator 110 may emit X-rays at a relatively low dose toward the breast 500 of the examination subject at a predetermined location, and the X-ray detector 120 may detect the X-rays that have passed through the breast 500 of the examination subject to acquire first image data. Referring to FIG. 2A, for example, the predetermined location may be a location P₀ above a center portion of the X-ray detector 120 with respect to the left-right direction of the examination subject. In addition, although not illustrated in FIG. 2A, the X-ray irradiator 110 may emit X-rays at any location other than the location P₀ to acquire the first image data, or may additionally emit X-rays at any other predetermined locations (e.g., positions P₋₂, P₋₁, P₁, P₂ of FIG. 2B or other locations) to additionally acquire the first image data.

For example, at least one of a first dose, voltage, current, exposure time, exposure interval, frequency, and wavelength for scout imaging may be controlled by the controller 200. Herein, the first dose may be a total dose of X-rays emitted from the X-ray irradiator 110 for scout imaging (scout scanning) to acquire the first image data. For example, at least one of the first dose, voltage, current, exposure time, exposure interval, frequency, and wavelength may be a predetermined value or may be adjusted by the user in real time. For example, when the X-ray irradiator 110 emits X-rays toward the examination subject at an arbitrary location to acquire the first image data, the first dose may mean the total amount of X-rays emitted by the X-ray irradiator 110 toward the examination subject to acquire the first image data at that one location. For example, when the X-ray irradiator 110 emits X-rays toward the examination subject at a plurality of locations in order to acquire the first image data, the first dose may mean the total amount of X-rays emitted by the X-ray irradiator 110 toward the examination subject to acquire the first image data at the plurality of locations. For example, the controller 200 may adjust the first dose by adjusting at least one of voltage, current, exposure time, exposure interval, frequency, and wavelength of X-rays.

The controller 200 may receive the first image data acquired by the X-ray detector 120 in response to the X-ray irradiator 110 emitting X-rays toward the breast 500 of the examination subject at the first dose at any one location and may generate a scout image (also called a preliminary image or reconnaissance image). In addition, the controller 200 may receive the first image data acquired by the X-ray detector 120 in response to the X-ray irradiator 110 emitting X-rays toward the breast 500 of the examination subject at the first dose at a plurality of locations and may reconstruct the first image data to generate a scout image. The controller 200 may transmit the scout image to the diagnosis portion 300. However, embodiments of the present disclosure are not limited thereto, and the X-ray detector 120 or the diagnosis portion 300 may directly generate the scout image.

The diagnosis portion 300 may analyze the first image data based on pre-training on breast image data categorized by type of breast lesion and may estimate type of breast lesion of the examination subject indicated in the first image data. For example, the diagnosis portion 300 may analyze the scout image based on pre-training on breast image data categorized by type of breast lesion and may estimate type of breast lesion of the examination subject indicated in the scout image. The estimated type of breast lesion may include mass, microcalcification, and other lesions other than mass and microcalcification. In addition, the diagnosis portion 300 may diagnose that it is “normal” or “undetected (no abnormality detected)” if breast lesion of the examination subject is not detected. The diagnosis portion 300 may transmit the result of the estimated type of breast lesion of the examination subject to the controller 200.

The controller 200 may determine an angular range within which the X-ray irradiator 110 moves (rotates) to perform DBT, according to the estimated type of breast lesion of the examination subject received from the diagnosis portion 300. For example, the X-ray irradiator 110 may perform DBT by emitting X-rays toward the breast of the examination subject at every predetermined angular interval while moving around the breast of the examination subject within the angular range determined according to the estimated type of breast lesion. However, embodiments of the present disclosure are not limited thereto, and the X-ray irradiator 110 and the X-ray detector 120 may be fixed, and the chamber in which the examination subject is located may move (rotate) with respect to the X-ray irradiator 110 and the X-ray detector 120 such that the X-ray irradiator 110 and the X-ray detector 120 may acquire an image, and in this case, the determined angular range may be the angular range in which the chamber moves (rotates).

In an embodiment, in a case where the estimated type of breast lesion of the examination subject is a mass (e.g., lump), DBT may be performed within a first angular range, and in a case where the estimated type of breast lesion of the examination subject is microcalcification, DBT may be performed within a second angular range, and the first angular range may be wider than the second angular range. That is, the first angular range may be greater than the second angular range. In addition, in a case where the estimated type of breast lesion of the examination subject is a lesion other than mass or microcalcification, or in a case where the estimated breast lesion is “undetected,” DBT may be performed within a predetermined angular range selected between the first angular range and the second angular range. Herein, the first angular range and the second angular range may be angles at which the X-ray irradiator 110 moves (rotates) around the breast of the examination subject, and this will be described in detail below with reference to FIGS. 2B-2C. For example, in a case where the estimated type of breast lesion of the examination subject is a mass (e.g., lump), the first angular range within which DBT is performed may be in a range of 40 to 60 degrees, for example, 50 degrees, and in a case where the estimated type of breast lesion of the examination subject is microcalcification, the second angular range within which DBT is performed may be in a range of 5 to 25 degrees, for example, 15 degrees. However, embodiments of the present disclosure are not limited thereto, and as long as the condition that the first angular range is wider than the second angular range is satisfied, the first angular range and the second angular range may be larger or smaller than the aforementioned angular range. In addition, in a case where the estimated type of breast lesion of the examination subject is other lesions or in a case where the no abnormality is detected, DBT may be performed within a predetermined angular range or within an angular range input in real time by the user's choice in addition to the first angular range and the second angular range. For example, in a case where the estimated type of breast lesion of the examination subject is “other lesions” or in a case where the estimated breast lesion is “undetected (no abnormality detected),” DBT may be performed in a third angular range that is smaller than the first angular range and greater than the second angular range.

In another embodiment, in a case where the estimated type of breast lesion of the examination subject is a mass (e.g., lump), DBT may be performed in the first angular range, and in a case where the estimated type of breast lesion of the examination subject does not include a mass and the mass is not detected, DBT may be performed in the second angular range, and the first angular range may be wider than the second angular range. For example, in a case where the estimated type of breast lesion of the examination subject is a mass, the first angular range within which DBT is performed may be in a range of 40 to 60 degrees (e.g., 50 degrees), and in a case where the estimated type of breast lesion of the examination subject is not a mass, the second angular range within which DBT is performed may be in a range of 5 to 25 degrees (e.g.,, 15 degrees). However, embodiments of the present disclosure are not limited thereto, and as long as the condition that the first angular range is wider than the second angular range is satisfied, the first angular range and the second angular range may be larger or smaller than the respective aforementioned angular range.

In an embodiment, in a case where the estimated type of breast lesion of the examination subject includes both mass and calcification, DBT may be performed within the first angular range or may be performed in a predetermined angular range selected between the first angular range and the second angular range. For example, the diagnosis portion 300 may estimate a probability value for each type of breast lesion of the examination subject, and the controller 200 may perform DBT within a predetermined angular range selected between the first angular range and the second angular range according to the estimated probability value. For example, in a case where the probability value for a mass is greater than or equal to a predetermined threshold value, DBT may be performed within the first angular range, and in a case where the probability value for a mass is less than the predetermined threshold value, DBT may be performed within the second angular range. However, embodiments of the present disclosure are not limited thereto, and in a case where the estimated type of breast lesion of the examination subject includes both mass and calcification, the user conducting the examination may be informed of the results of the estimated type of breast lesion of the examination subject, and an angular range for performing DBT may be determined according to the user's input. For example, in a case where the estimated type of breast lesion of the examination subject includes both mass and calcification, DBT may be performed within a third angular range that is smaller than the first angular range and greater than the second angular range.

FIG. 2B is a view schematically illustrating a process in which the X-ray irradiator 110 and the X-ray detector 120 perform DBT on the examination subject within the first angular range in a case where the estimated type of breast lesion estimated by the diagnosis portion 300 includes, for example, a mass.

Referring to FIGS. 1 and 2B, in an embodiment, the X-ray irradiator 110 may perform DBT by emitting X-rays toward the breast 500 of the examination subject while moving (rotating) within the first angular range (50° in FIG. 2B) determined by the controller 200 to acquire the second image data. That is, in FIG. 2B, the angular range determined according to the estimated type of breast lesion may be 50° as the first angular range. Herein, the X-ray irradiator 110 may move (rotate) along an elliptical arc or a circular arc, but embodiments of the present disclosure are not limited thereto, and the X-ray irradiator 110 may also move in a straight line, curve, or irregularly within the first angular range.

For example, the X-ray irradiator 110 may emit X-rays toward the breast 500 of the examination subject at a position P₀ above a center portion of the X-ray detector 120 with respect to the left-right direction of the examination subject, at positions P₋₂, P₂ rotated by a predetermined angle (−25°, 25° in FIG. 2B) in a counterclockwise direction and a clockwise direction, respectively, with respect to the position P₀, and at positions P₋₁, P₁ between the position P₀ and each position P₋₂, P₂ to acquire second image data. That is, the X-ray irradiator 110 may rotate clockwise and counterclockwise with respect to the position P₀ within the angular range (e.g., 50° in FIG. 2B) determined by the controller 200.

In addition, the X-ray irradiator 110 may emit X-rays toward the breast 500 of the examination subject at every predetermined angular interval (12.5° in FIG. 2B), between the position P₀ and the positions P₋₂, P₂, respectively, to acquire the second image data. Herein, the predetermined angular interval (12.5° in FIG. 2B) may be set in advance based on the predetermined angular range (50° in FIG. 2B), that is, the angular range determined by the controller 200 according to the estimated type of breast lesion, and may also be adjusted in real time according to the user's input. In FIG. 2B, it is illustrated that the positions P₋₁, P₁ between the position P₀ and the positions P₋₂, P₂, respectively, are a single pair, but this is for convenience of explanation, and the positions P₋₁, P₁ where the X-ray irradiator 110 emits X-rays to the breast 500 of the examination subject between the positions P₀ and the positions P₋₂, P₂ may include a plurality of pairs of positions.

For example, the X-ray irradiator 110 may emit X-rays toward the breast 500 at every predetermined angular interval while sequentially moving from the position P₋₂ to the position P₋₁, the position P₀, the position P₁, and then the position P₂ to acquire the second image data. However, embodiments of the present disclosure are not limited thereto, and the X-ray irradiator 110 may emit X-rays at every predetermined angular interval while sequentially moving from the position P₂ to the position P₁, the position P₀, the position P₋₁, and then the position P₋₂ to acquire second image data. For example, in a case where the angular range (50° in FIG. 2B) within which the X-ray irradiator 110 is to move and the positions (P₋₂, P₋₁, P₀, P₁, P₂ in FIG. 2B) at which the X-ray irradiator 110 is to emit X-rays are determined according to the estimated type of breast lesion, the controller 200 may move the X-ray irradiator 110 to the X-ray emission start position, for example, P₋₂ or P₂.

Although it is illustrated in FIG. 2B that the X-ray irradiator 110 emits X-rays at positions P₋₂, P₋₁, P₀, P₁, P₂ to acquire image data, embodiments of the present disclosure are not limited thereto, and the X-ray irradiator 110 may emit X-rays at any position set in advance based on the first angular range determined by the controller 200 according to the estimated type of breast lesion to acquire second image data. For example, the position where the X-ray irradiator 110 emits X-rays may deviate from the reference position P₀, and the second image data may be acquired by emitting X-rays from even-numbered, symmetrical positions in the left-right direction, instead of odd-numbered positions.

FIG. 2C is a view schematically illustrating a process in which the X-ray irradiator 110 and the X-ray detector 120 perform DBT on the examination subject within the second angular range in a case where the estimated type of breast lesion estimated by the diagnosis portion 300 is, for example, microcalcification or other lesions, or does not include a mass.

Referring to FIGS. 1 and 2C, in an embodiment, the X-ray irradiator 110 may perform DBT by emitting X-rays toward the breast 500 of the examination subject at every predetermined interval while moving within the second angular range (15° in FIG. 2C) determined by the controller 200 to acquire the second image data. That is, in FIG. 2C, the angular range determined according to the estimated type of breast lesion may be 15° as the second angular range. Herein, the X-ray irradiator 110 may move (rotate) along an elliptical arc or a circular arc, but embodiments of the present disclosure are not limited thereto, and the X-ray irradiator 110 may move in a straight line, curve, or irregularly within the second angular range.

For example, the X-ray irradiator 110 may emit X-rays toward the breast 500 of the examination subject at a position P₀ above a center portion of the X-ray detector 120 (e.g., a center portion of the breast 500 of the examination subject) and positions P₋₁, P₁ rotated by a predetermined angle (−7.5°, 7.5° in FIG. 2C) in a counterclockwise direction and a clockwise direction, respectively, with respect to the position P₀ to acquire the second image data. That is, the X-ray irradiator 110 may rotate clockwise and counterclockwise with respect to the position P₀ within the angular range (15° in FIG. 2C) determined by the controller 200.

Although not illustrated in FIG. 2C, the X-ray irradiator 110 may emit X-rays toward the breast 500 of the examination subject at certain angular intervals between the position P₀ and the position P₋₁, P₁ to additionally acquire the second image data. Herein, the certain angular interval may be set in advance based on the angular range determined by the controller 200 according to the estimated type of breast lesion, and may also be adjusted in real time according to the user's input. For example, the positions at which the second image data is acquired between the position P₀ and the position P₋₁, P₁ may include a plurality of pairs of positions.

For example, the X-ray irradiator 110 may emit X-rays toward the breast 500 of the examination subject at every predetermined interval while sequentially moving from position P₋₁ to position P₀ and then position P₁ to acquire the second image data. However, embodiments of the present disclosure are not limited thereto, and the X-ray irradiator 110 may emit X-rays toward the breast 500 of the examination subject at every predetermined interval while sequentially moving from position P₁ to position P₀ and then position P₋₁ to acquire the second image data. For example, in a case where the angular range (15° in FIG. 2C) within which the X-ray irradiator 110 is to move and the positions (P₋₁, P₀, P₁ in FIG. 2C) at which the X-ray irradiator 110 is to emit X-rays are determined according to the estimated type of breast lesion, the controller 200 may move the X-ray irradiator 110 to the X-ray emission start position, for example, P₋₁ or P₁.

Although it is illustrated in FIG. 2C that the X-ray irradiator 110 emits X-rays at positions P₋₁, P₀, P₁ to acquire the second image data, embodiments of the present disclosure are not limited thereto, and the X-ray irradiator 110 may emit X-rays at any arbitrary position set in advance based on the second angular range determined by the controller 200 according to the estimated type of breast lesion to acquire the second image data. For example, the position at which the X-ray irradiator 110 emits X-rays may deviate from the reference position P₀, and X-rays may be emitted at even-numbered, left-right symmetrical positions instead of odd-numbered position to acquire the second image data.

Referring to FIGS. 2B and 2C, it may be appreciated that the first angular range (50° in FIG. 2B) within which DBT is performed in a case where the estimated type of breast lesion of the examination subject includes a mass is wider than the second angular range (15° in FIG. 2C) within which DBT is performed in a case where the estimated type of breast lesion of the examination subject includes microcalcification or other lesions, or does not include a mass.

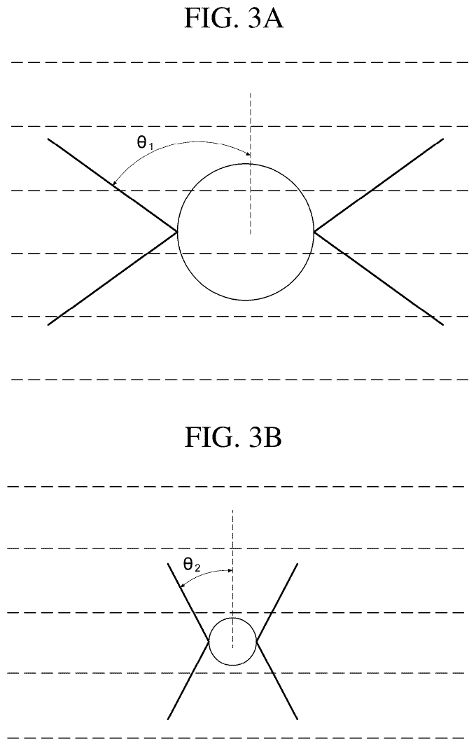

FIG. 3A is a view for explaining a process in which a first lesion type is imaged (e.g., scanned) within the first angular range according to an embodiment of the present disclosure. FIG. 3B is a view for explaining a process in which a second lesion type is imaged within the second angular range according to an embodiment of the present disclosure. For example, FIG. 3A illustrates that DBT is performed within a first angular range θ1 in a case where the estimated type of breast lesion of the examination subject is a first lesion type including a mass, and FIG. 3B illustrates that DBT is performed within a second angular range θ2 in a case where the estimated type of breast lesion of the examination subject is a second lesion type including microcalcification or other lesion, or not including a mass, where the first angular range θ1 is wider than the second angular range θ2.

Referring again to FIGS. 1, 2B and 2C, in an embodiment, at least one of a second dose, voltage, current, exposure time, exposure interval, frequency, and wavelength of X-rays to perform DBT to acquire the second image data may be controlled by the controller 200. Herein, the second dose may be a total dose of X-rays emitted from the X-ray irradiator 110 to perform DBT to acquire the second image data.

For example, the controller 200 may perform automatic exposure control (AEC) for X-rays emitted in the DBT before performing the DBT to acquire the second image data as in FIGS. 2B and 2C, based on the first image data acquired in the scout imaging as in FIG. 2A (or the scout image generated based on the first image data).

For example, the controller 200 may determine at least one of the second dose voltage (the total dose), current, exposure time, exposure interval, frequency, and wavelength of X-rays emitted from the X-ray irradiator 110 to perform DBT to acquire the second image data based on the first image data or the scout image generated based thereon. However, embodiments of the present disclosure are not limited thereto, and at least one of the second dose, voltage, current, exposure time, exposure interval, frequency, and wavelength of X-rays emitted from the X-ray irradiator 110 may be a predetermined value or may be input by the user in real time. For example, in a case where the X-ray irradiator 110 emits X-rays toward the examination subject at a plurality of locations to acquire the second image data, the second dose may mean the total amount of X-rays emitted by the X-ray irradiator 110 toward the examination subject to acquire the second image data at the plurality of locations. For example, the controller 200 may adjust the second dose by adjusting at least one of the voltage, current, exposure time, exposure interval, frequency, and wavelength of the X-rays.

In an embodiment, the first dose, which is the total amount of X-rays emitted by the X-ray irradiator 110 to acquire the first image data by performing scout imaging, may be less than the second dose, which is the total amount of X-rays emitted by the X-ray irradiator 110 to acquire the second image data by performing DBT. For example, the total dose of the first dose in performing scout imaging may be 50% or less, specifically 30% or less, and more specifically 10% or less, of the total dose of the second dose in performing DBT, but embodiments of the present disclosure are not limited thereto, and the percentage may be larger than that.

The controller 200 may receive the second image data from the imaging portion 100 and reconstruct the second image data to generate a tomosynthesis image. For example, the X-ray irradiator 110 may emit X-rays toward the examination subject at predetermined locations, and the X-ray detector 120 may detect X-rays that have passed through the examination subject to acquire the second image data, and the controller 200 may receive the second image data and perform reconstruction to generate and output a tomosynthesis image.

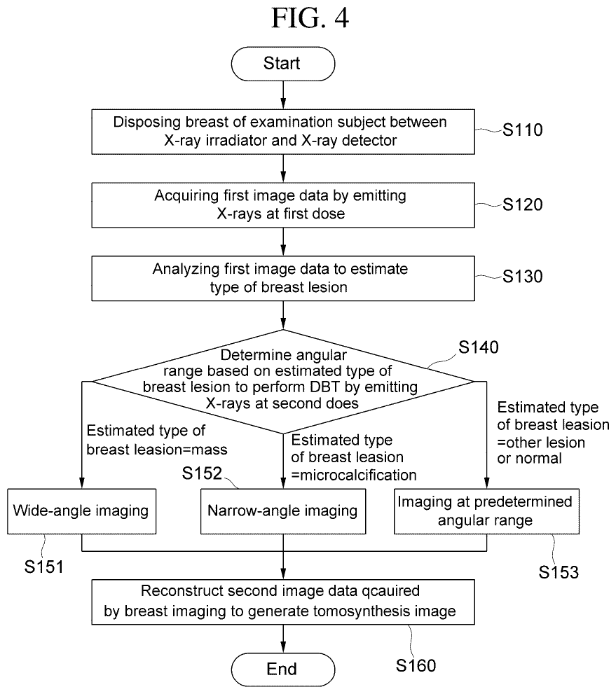

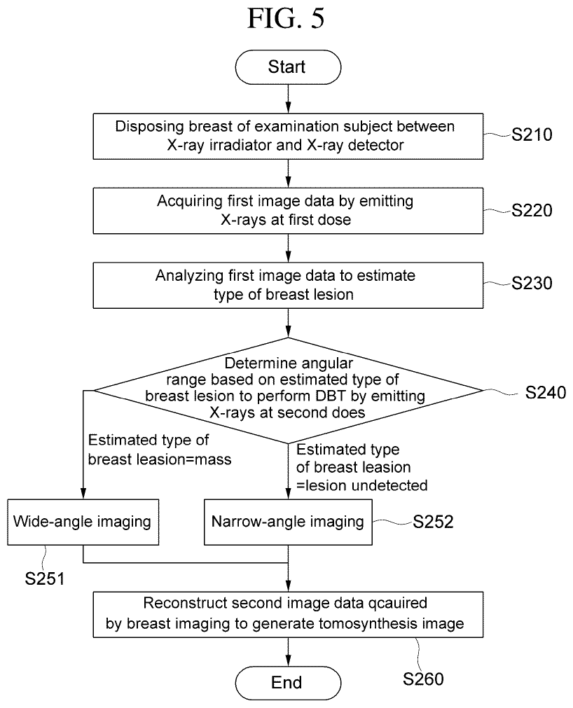

Hereinafter, with reference to FIGS. 4-5, a DBT method according to an embodiment of the present disclosure will be described.

FIG. 4 is a flowchart illustrating a DBT method according to an embodiment of the present disclosure.

According to the DBT method according to an embodiment of the present disclosure, the breast of the examination subject may be disposed between the X-ray irradiator and the X-ray detector (S110). For example, the breast of the examination subject may be compressed by a pair of compression plates.

Next, the X-ray irradiator may emit X-rays toward the breast of the examination subject such that the imaging portion acquires first image data (S120). For example, the breast of the examination subject may be irradiated with X-rays emitted from the X-ray irradiator. The first image data may also be referred to as scout image data, preliminary image data, or reconnaissance image data. In an embodiment, the X-ray irradiator may emit X-rays at a first dose toward the breast of the examination subject at a predetermined location, and the X-ray detector may detect the X-rays that have passed through the breast of the examination subject to acquire the first image data. Herein, the first dose may be a low dose for scout imaging. The predetermined location may be a location above a center portion of the X-ray detector 120 with respect to the left and right direction of the examination subject. However, embodiments of the present disclosure are not limited thereto, and the predetermined location may be at least one arbitrary location.

Next, the first image data may be analyzed using at least one of an AI-based diagnostic module or an artificial neural network, which is pre-trained based on breast image data categorized by breast lesion type, to estimate type of breast lesion of the examination subject (S130). Here, the estimated type of breast lesion may include mass, microcalcification, and other lesions other than mass and microcalcification. For example, the pre-trained AI image diagnosis portion and artificial neural network may use at least one of an AI image diagnosis module, a computer-aided diagnosis (CAD) module, an artificial neural network, a machine learning network, a deep learning network, a machine learning/deep learning classification model, and a machine learning/deep learning regression model. In addition, at least one of the AI image diagnosis module and the artificial neural network may be trained to receive the first image data and estimate type of breast lesion of the examination subject based on the first image data.

Next, an angular range for performing DBT may be determined according to the estimated type of breast lesion (S140). For example, in a case where the estimated type of breast lesion of the examination subject is a mass, the determined angular range may be a first angular range, in a case where the estimated type of breast lesion of the examination subject is microcalcification, the determined angular range may be a second angular range, and in a case where the estimated type of breast lesion of the examination subject is a lesion other than mass and microcalcification, the determined angular range may be a predetermined angular range selected between the first angular range and the second angular range.

Next, DBT may be performed in which the X-ray irradiator rotates around the breast of the examination subject within the determined angular range and emits X-rays toward the breast of the examination subject at every predetermined angular interval to acquire second image data for a plurality of angles. For example, in a case where the estimated type of breast lesion is a mass, the imaging portion may perform DBT within the first angular range (S151), in a case where the estimated type of breast lesion is microcalcification, the imaging portion may perform DBT within the second angular range (S152), and in a case where the estimated type of breast lesion is a lesion other than mass and microcalcification, the imaging portion may perform DBT in a predetermined angular range selected between the first angular range and the second angular range (S153). In such a case, the first angular range may be wider than the second angular range, and the second angular range may be narrower than the first angular range. For example, the first angular range may be in a range of 40 to 60 degrees, and the second angular range may be in a range of 5 to 25 degrees.

In an embodiment, a first dose, which is a total X-ray amount emitted in acquiring the first image data, may be less than a second dose, which is a total X-ray amount emitted in performing DBT. For example, the first dose, which is the total X-ray dose in acquiring the first image data, may be 50% or less, specifically 30% or less, and more specifically 10% or less, of the second dose, which is the total X-ray dose in performing DBT, but embodiments of the present disclosure are not limited thereto and the percentage may be more than that.

In an embodiment, automatic exposure control (AEC) for X-rays emitted in performing DBT may be performed based on the first image data. For example, at least one of the second dose, voltage, current, exposure time, exposure interval, frequency, and wavelength may be determined based on the first image data.

Next, the second image data for the plurality of angles may be reconstructed to generate a tomosynthesis image (S160).

FIG. 5 is a flowchart illustrating a DBT method according to another embodiment of the present disclosure.

According to a DBT method according to another embodiment of the present disclosure, the breast of the examination subject may be disposed between the X-ray irradiator and the X-ray detector (S210). For example, the breast of the examination subject may be compressed by a pair of compression plates.

Next, the X-ray irradiator may emit X-rays toward the breast of the examination subject such that the imaging portion may acquire first image data (S220). For example, the breast of the examination subject may be irradiated with X-rays emitted from the X-ray irradiator. The first image data may also be referred to as scout image data, preliminary image data, or reconnaissance image data. In an embodiment, the X-ray irradiator may emit X-rays at a first dose toward the breast of the examination subject at a predetermined location, and the X-ray detector may detect X-rays that have passed through the breast of the examination subject to acquire the first image data. Herein, the first dose may be a low dose for scout imaging. The predetermined location may be a location above a center portion of the X-ray detector (e.g., a center portion above the breast of the examination subject). However, embodiments of the present disclosure are not limited thereto, and the predetermined location may be at least one arbitrary location.

Next, the first image data may be analyzed using at least one of an AI-based diagnostic module or an artificial neural network, which is pre-trained based on breast image data categorized by breast lesion type, to estimate type of breast lesion of the examination subject (S230). Here, the estimated type of breast lesion may include mass, microcalcification, and other lesions other than mass and microcalcification. For example, the pre-trained AI image diagnosis portion and artificial neural network may use at least one of an AI image diagnosis module, a computer-aided diagnosis (CAD) module, an artificial neural network, a machine learning network, a deep learning network, a machine learning/deep learning classification model, and a machine learning/deep learning regression model. In addition, at least one of the AI image diagnosis module and the artificial neural network may be trained to receive the first image data and estimate type of breast lesion of the examination subject based on the first image data.

Next, an angular range for performing DBT may be determined according to the estimated type of breast lesion (S240). For example, in a case where the estimated type of breast lesion of the examination subject is a mass, the determined angular range may be a first angular range, and in a case where the estimated type of breast lesion of the examination subject does not include a mass and the mass is undetected, the determined angular range may be a second angular range.

Next, DBT may be performed in which the X-ray irradiator rotates around the breast of the examination subject within the determined angular range and emits X-rays toward the breast of the examination subject at every predetermined angular interval to acquire second image data for a plurality of angles. For example, in a case where the estimated type of breast lesion is a mass, the imaging portion may perform DBT within the first angular range (S251), and in a case where the estimated type of breast lesion does not include a mass and the mass is undetected, the imaging portion may perform DBT within the second angular range (S252). In such a case, the first angular range may be wider than the second angular range, and the second angular range may be narrower than the first angular range. For example, the first angular range may be in a range of 40 to 60 degrees, and the second angular range may be in a range of 5 to 25 degrees.

In an embodiment, a first dose, which is a total X-ray amount emitted in acquiring the first image data, may be less than a second dose, which is a total X-ray amount emitted in performing DBT. For example, the first dose, which is the total X-ray dose in acquiring the first image data, may be 50% or less, specifically 30% or less, and more specifically 10% or less, of the second dose, which is the total X-ray dose in performing DBT, but embodiments of the present disclosure are not limited thereto and the percentage may be more than that.

In an embodiment, automatic exposure control (AEC) for X-rays emitted in performing DBT may be performed based on the first image data. For example, at least one of the second dose, voltage, current, exposure time, exposure interval, frequency, and wavelength may be determined based on the first image data.

Next, the second image data for the plurality of angles may be reconstructed to generate a tomosynthesis image (S260).

As such, according to an embodiment of the present disclosure, the angular range for DBT may be determined patient-specifically, and DBT may be performed in an angular range more suitable for each patient. In addition, according to an embodiment of the present disclosure, the angular range of patient-customized DBT may be determined based on data acquired during the scout imaging process.

Although embodiments of the present disclosure have been described above with reference to the accompanying drawings, it will be understood by those skilled in the art that the technical concept of the present disclosure may be embodied in other specific forms without changing the technical spirit or essential characteristics of the invention.

Therefore, the embodiments described above should be considered illustrative in all respects and not restrictive. The scope of the invention is indicated by the claims set forth below rather than the foregoing detailed description, and all modifications or alterations derived from the meaning, scope, and equivalents of the claims are to be construed as being included within the scope of the present invention.

What is claimed is:

1. A digital breast tomosynthesis (DBT) method, comprising:
disposing a breast of an examination subject between an X-ray irradiator and an X-ray detector;
acquiring first image data by emitting X-rays by the X-ray irradiator toward the breast of the examination subject;
analyzing the first image data to estimate a type of breast lesion of the examination subject by using at least one of an artificial intelligence (AI) image diagnosis portion and an artificial neural network pre-trained based on breast image data categorized by breast lesion type;
determining an angular range for performing DBT according to the estimated type of breast lesion;
performing the DBT in which the X-ray irradiator rotates around the breast of the examination subject within the determined angular range and emits X-rays toward the breast at every predetermined angular interval, to acquire second image data corresponding to a plurality of angles; and
reconstructing the second image data acquired at the plurality of angles to generate a tomosynthesis image,
wherein a first dose, which is a total X-ray dose used in acquiring the first image data, is smaller than a second dose, which is a total X-ray dose used in performing the DBT.

2. The DBT method of claim 1,
wherein in a case where the estimated type of breast lesion of the examination subject is a mass, the determined angular range is a first angular range,
in a case where the estimated type of breast lesion of the examination subject is microcalcification, the determined angular range is a second angular range,
in a case where the estimated type of breast lesion of the examination subject is a lesion other than mass or microcalcification, or where the estimated type of breast lesion of the examination subject is undetected, the determined angular range is a predetermined angular range selected between the first angular range and the second angular range, and
the first angular range is wider than the second angular range.

3. The DBT method of claim 2,
wherein the first angular range is in a range of 40 to 60 degrees, and
the second angular range is in a range of 5 to 25 degrees.

4. The DBT method of claim 1,
wherein in a case where the estimated type of breast lesion of the examination subject is a mass, the determined angular range is a first angular range,
in a case where a mass is undetected in the estimated type of breast lesion of the examination subject, the determined angular range is a second angular range, and
the first angular range is wider than the second angular range.

5. The DBT method of claim 1, further comprising:
performing automatic exposure control (AEC) for the X-rays emitted during the performing the DBT, based on the first image data.

6. The DBT method of claim 1, further comprising:
determining at least one of the second dose, voltage, current, and exposure time of the X-rays emitted during the performing the DBT, based on the first image data.

7. The DBT method of claim 1,
wherein the first dose is 50% or less of the second dose.

8. The DBT method of claim 1,
wherein the at least one of the pre-trained AI image diagnosis portion and the pre-trained artificial neural network includes at least one of an AI image diagnosis module, a computer-aided diagnosis (CAD) module, a machine learning network, a deep learning network, a machine learning/deep learning classification model, and a machine learning/deep learning regression model.

9. The DBT method of claim 1,
wherein the at least one of the pre-trained AI image diagnosis portion and the pre-trained artificial neural network is trained to receive the first image data and estimate the type of breast lesion of the examination subject based on the first image data.

10. A digital breast tomosynthesis (DBT) system comprising:
an X-ray irradiator;
an X-ray detector disposed to face the X-ray irradiator;
a memory configured to store computer-readable instructions for controlling the X-ray irradiator and the X-ray detector to generate a tomosynthesis image; and
a processor electrically connected to the memory and implemented to execute the instructions,
wherein the processor, upon executing the instructions, is configured to:
acquire first image data by the X-ray irradiator emitting X-rays toward the breast of the examination subject disposed between the X-ray irradiator and the X-ray detector;
analyze the first image data to estimate a type of breast lesion of the examination subject by using at least one of an artificial intelligence (AI) image diagnosis portion and an artificial neural network pre-trained based on breast image data categorized by breast lesion type;
determine an angular range for performing DBT according to the estimated type of breast lesion;
perform the DBT in which the X-ray irradiator rotates around the breast of the examination subject within the determined angular range and emits X-rays toward the breast at every predetermined angular interval, to acquire second image data corresponding to a plurality of angles; and
reconstruct the second image data acquired at the plurality of angles to generate a tomosynthesis image, and
a first dose, which is a total X-ray dose used in acquiring the first image data, is smaller than a second dose, which is a total X-ray dose used in performing the DBT.

11. The DBT system of claim 10,
wherein in a case where the estimated type of breast lesion of the examination subject is a mass, the determined angular range is a first angular range,
in a case where the estimated type of breast lesion of the examination subject is microcalcification, the determined angular range is a second angular range,
in a case where the estimated type of breast lesion of the examination subject is a lesion other than mass or microcalcification, or where the estimated type of breast lesion of the examination subject is undetected, the determined angular range is a predetermined angular range selected between the first angular range and the second angular range, and
the first angular range is wider than the second angular range.

12. The DBT system of claim 10,
wherein in a case where the estimated type of breast lesion of the examination subject is a mass, the determined angular range is a first angular range,
in a case where a mass is undetected in the estimated type of breast lesion of the examination subject, the determined angular range is a second angular range, and
the first angular range is wider than the second angular range.

13. The DBT system of claim 10,
wherein the processor is further configured to perform automatic exposure control (AEC) for the X-rays emitted during the performing the DBT, based on the first image data.

14. The DBT system of claim 10,
wherein the processor is further configured to determine at least one of the second dose, voltage, current, and exposure time of the X-rays emitted during the performing the DBT, based on the first image data.
